(19) United States
(12) Patent Application Publication
(10) Pub. No.: US 2015/0157235 A1
(43) Pub. Date: Jun. 11, 2015

(54) Public Health Screening For Cognitive Health Using Dry Sensor Electroencephalogram Technology

(71) Applicants: James J. Jelen, Glen Ellyn, IL (US); Ajeet S. Charate, Plainfield, IL (US); Leonid Levin, Palatine, IL (US)

(72) Inventors: James J. Jelen, Glen Ellyn, IL (US); Ajeet S. Charate, Plainfield, IL (US); Leonid Levin, Palatine, IL (US)

(21) Appl. No.: 14/100,454

(22) Filed: Dec. 9, 2013

(51) Int. Cl. A61B 5/0478; A61B 5/00; G01R 33/563; G01R 33/48; A61B 6/03; A61B 5/16; A61B 5/0482

(52) U.S. Cl. CPC A61B 5/0478; A61B 5/168; A61B 5/4088; A61B 5/4082; A61B 5/4824; A61B 5/0482; A61B 5/4848; A61B 5/4094; A61B 5/165; A61B 5/4806; G01R 33/4806; A61B 6/032; A61B 6/037; G01R 33/56341

(57) ABSTRACT

Systems and methods are provided for performing neurometric evaluation of Quantitative Electro Encephalogram (QEEG) data, derived from Dry Sensor technology, as opposed to the use of any types of conventional paste/gel and silver/silver chloride sensors. The individuals who would be helped by these screening procedures are identified.

### CROSS REFERENCE TO RELATED APPLICATIONS

Follow up to provisional patent No. 61/735,065 filed on Dec. 10, 2012. The title of the provisional patent application is also used in this patent application.

### FEDERALLY SPONSORED RESEARCH OR DEVELOPMENT

NOT APPLICABLE

### BACKGROUND OF THE INVENTION

Quantitative electroencephalographic analysis (also called neurometric analysis) has been used for many years to help identify and assist with the assessment, diagnosis and treatment of a myriad of neuropsychiatric conditions. (See US 2009/0312663 A1, E. Roy John). Epilepsy evaluation, traumatic head injuries, response to drug treatments and sleep disorders are just a few these conditions where the QEEG can be a useful tool. The Food and Drug Administration has granted QEEG approval as a Class II device (see K974748), and a number of commercial systems are available.

Typically, when an individual attends a QEEG recording session, they follow a protocol of having a cap with nineteen electrodes fitted on their head, and the application of a conductive gel is applied on each electrode to allow for an improved connection to the scalp. The gel decreases the characteristic impedance of the sensors to approximately 5000 ohms or less and allows for slight movements of the head to generate only a small amount of electrical noise and smooth out voltage spikes, called artifacts. Data can then be collected for 10-20 minutes at pre-determined sampling rate (typically 128-256 samples per second), post processed to remove other recorded noise, note and correct any significant processing issues, and then be quantified and analyzed. Analysis will normally include a power spectrum, frequency data and phase interrelationships between specific electrode locations. These statistics can then be compared to a normalized or reference group (age and sex matched), with the goal of highlighting deviations from a normal distribution that point to potential dysfunctions.

There exist no less than three major commercial databases available to health therapists and clinicians to compare QEEG data sets and profile the record based upon the scalp recordings: (HBI-St. Petersburg, Russia, Neuroguide QEEG of St. Petersburg, Fla., and NxLink-New York University, New York).

There are also many other special purpose, task specific EEG systems and databases, which may only use part of the complete EEG sensor set from the International 10-20 System. Here are some examples.
1) An ADHD diagnostic data set to quickly diagnose at least one subtype of attention deficit disorder (US 2011/0066065). This specific tool uses only a few electrodes to collect data and generate a recommendation.
2) A diagnostic EEG test developed at the Boston Children's University Hospital by Frank Duffy and Heidelise Als. This diagnostic test evaluates autism risks for very young children.
3) A portable device, (EEG in a Bag), developed at the New York University for the use of quick diagnosis of epilepsy conditions, or for patients who are unconscious.
   Imec Group of Belgium, an eight sensor headset used primarily for assessment of orthopedic and pain issues.
4) The Brainwave Biofeedback (Neurofeedback) community has been using 2 to 4 probe EEG assessments for years (also called “Mini-Qs”) to look for basic diagnostic information related to sensorimotor functioning, anxiety, and sleep, among other issues.

### Dry Sensor Technology

As the QEEG class databases have grown and analysis techniques have improved, so has the sensor hardware technology. As a partial solution to the cumbersome and inconvenient method of using pastes or gels inside of silver chloride electrodes, active sensor using little or no conductive supplements are now becoming more common in the use of brain computer interfaces (BCI). Some of the sensors use saline solutions as a conductive (ion) medium to the scalp, or, in a few cases, metal contacts which press against the scalp. Another variant is the use of electrically “active” sensor, utilizing an electrically powered amplifier to increase the voltage of the sensed EEG signal. IMEC, of Belgium (Patent #8454505 B2, 2013) has developed a hardware based biopotential sensor for EEG and EMG that attenuates motion artifacts to record very low signal to noise signals of high quality. There are other methods that include skin screws, organic conductive polymers, bristled brush systems, and micro machined sensing surfaces to the skin. And finally Quantum Applied Science and Research has developed an insulated bioelectrode (IBE) that gives very good results using a circuit with capacitive coupling and impedance transformation to sense electric potentials on the skin. (U.S. Pat. No. 66,961,601).

Many of these new active sensors have shown very good signal quality, artifact management and repeatability. In many research cases they are comparable to conventional EEG acquisition techniques. In consort with better sensor acquisition of the analog portion of the EEG sampled signals, improved software algorithms and processing has complimented the new hardware to improve signal to noise levels. Software has also been used to perform more sophisticated post processing signal component analysis of the collected signals, optimizing and improving the tasks of artifact removal and the removal of fixed pattern noise sources. However more dry sensor data should be collected and compared with conventional systems so that the validation of data integrity is confirmed, and the differentiation of which techniques and measurements yield the most effective data are understood fully.

### Dry Sensor Risks

In general there is very little physical risk to the subject in the process of collecting this new form of EEG data. The use of an active (that is, electrically powered) electrodes requires an additional power supply. Some types of dry sensors may use hardened substrates that may be uncomfortable on the scalp, or brush or bristle mechanical arrangements that range from slightly uncomfortable to possibly painful when attached securely. There may also be some pre testing skin preparations to optimize electrical contact that may irritate the scalp on the areas they touch. Finally, the person performing the administration of the testing will require specialized, but not extensive training to insure client safety and collect optimal results.

The low risk and relative convenience of using dry sensor EEG diagnostic testing make it ideal for large scale screening of at risk populations. The results may also be used in conjunction with other medical tools such as imaging (MRI, DTI, CT scanning) or LORETA (Low Resolution Electromagnetic Tomography) to provide more complete assessment, at low cost per unit of use.

### CONCLUSION

As these sensors can be safely used to speed up collection of EEG data, they could be leveraged to help assist in the public health venues where early detection of neurological, medical, mental and learning problems for large numbers of individuals, and especially children with correctable developmental conditions could lead to greater preventative or early intervention care initiatives. The ability to compare data results to a reference population will improve identification of abnormal conditions.

At the database level, a dry electrode technology should be used to generate large and specific age and sex matched data for use of normative comparisons and diagnosis. Normative database development and validation techniques are well accepted science and can also be leveraged to generate normative data as part of a rigorous diagnostic system.

### BRIEF SUMMARY OF THE INVENTION

A system, for use in the Public Health Community, (school systems, military organizations, research organizations such as Universities, or private research companies, state and local public health entities, national organizations for specific issues such as Autism Spectrum Disorders, Dementia, Fetal Alcohol Spectrum Disorder, Epilepsy), comprised of an Electroencephalogram recorder with between four and nineteen sensors, a headset which utilizes dry electrode (also called active probe) sensors, and computer software for performing QEEG analysis for the data collected. The dry sensor headset has an advantage of rapid and easy attachment to the client, and very good signal to noise quality when compared to conventional silver silver/chloride gel electrodes used in most EEG applications. Using this data collection method, a clients QEEG data variables (such as frequency, spectral power, statistical Z score, phase correlation) can be compared to a normalized data base of other individuals of the same sex and approximate age and then assessed for abnormalities based on statistical deviation from the database standards. Individuals may then be identified for possible follow up services, if indicated, and subsequent reevaluation for post treatment changes in brain functioning.

### DETAILED DESCRIPTION OF THE INVENTION

The present invention is positioned to collect and analyze electroencephalogram data from subjects using dry electrode sensors. The use of dry electrode sensors provides an easier and faster method for performing a diagnostic EEG than is available with conventional gel/paste type sensors. The invention design requires a calibration of the system before testing, baseline readings of system noise floor and artifact profiling. At acquisition time, the system stores the collected raw EEG data, organizes them into frequency bins and power spectrum tables, compares phase relationships among the signals collected and displays them in topographic fashion. The collected data can then be compared statistically to a normative database, and assigned relative and absolute scores for the purpose of assessing the subject for neurophysiological health, both as a direct measurement and as an adjunct to other methods of screening, such as classical psychometric testing. At the present time there are no known normative QEEG systems derived from dry sensor technology. However this limitation may only be temporary. It is conceivable that the data collected through this system can be characterized and integrated into internally developed databases that can be validated for diagnostic utility.

In order to insure a high level of measurement accuracy and repeatability the issues of artifact control and other sources of data noise will be carefully managed. A large number of laboratory studies have shown high correlation of the signal quality between dry and wet sensors, but there have not been large amounts of clinical data to suggest that the quality extends to non-experimental participants. This system, through a calibration of the signal path prior to use, through extensive signal processing methods, through montage selection with regard to specific modes of testing and finally through validation assessment when compared to conventional wet sensors, will provide means for a more efficacious type of EEG assessment than presently exists.

With regard to neurofeedback applications, the methods and system described here shall be used for diagnostic/evaluative purposes, or to generate normative data only. The real time processing and feedback of brainwave data is critically dependent on the latency time between the computer interface and the subject. There have been large variances of signal quality in the many types of dry sensors currently in use, so much so that any specific system cannot be certified to be deterministic to the level of where it is appropriate for neurofeedback training.

As shown in FIG. 1, the system of the present invention shows an embodiment consisting of three dry sensor channels (101) attached frontally to active amplifiers (102) for a QEEG collection. However, depending on the situation and test requirement, as many as 19 electrodes may be used. In addition to the active channels, other reference electrodes shall be used which will record muscle artifact, and electrocardiogram (EKG) response. Each channel is amplified by the active probes, converting the RAW EEG analog voltage into digital signals (102,103), and then transformed into a power spectrum using the Fast Fourier Transform technique (104). After the power spectrum has been post processed and characterized in terms of the variables of interest, it is ready for assessment. The FFT Power Spectrum is then compared to a QEEG database of a population of similar subjects in age, but also included to represent a Gaussian distribution of normality (105). Additionally, the raw data can be processed for measurements related to coherence and asymmetry. There are a number of commercially available systems suitable for the task of the EEG collection. For example, presently the QUASAR DSI 10/20 system is available with up to 12 selectable channels in the International 10/20 system. Imec, Hoist Centre of Leuven, Belgium also has an 8 channel active EEG headset available, which includes a wireless transmit mode. And also the g.tec medical engineering group of Austria has an 8 channel device using g.Sahara active electrodes.

FIG. 2 shows a diagram of a subset of the 10/20 System, with ellipses designating locations that would be required to provide for the analysis of a targeted array of conditions, such as Attention Deficit Hyperactivity Disorder (ADHD) or Autism. In a system with less than 19 available channels, these centrally located locations (201) (Fz, Cz, Pz, F3, F4, O1) are generally less affected by movement artifacts. Locations Fp1 and FP2 are more susceptible to blinking and eye twitches, but they are also very important for a number of prefrontal EEG signals involved with executive functioning and control. A screening diagnosis for ADHD has been performed using the Cz location by a system developed byNEBA Health LLC of Augusta, Ga. However this system uses conventional gel sensors. Boston Children's Hospital has also done preliminary work on spectral coherence measurements of specific sites to on the brain to distinguish so called “neuro-typical” brains from those that may become autistic.

Depending on the number of available channels other locations can be selected by the clinician, as is shown in the selection of secondary channel locations (sites F7/F8, C3/C4, P3/P4). In the case of the earlier mentioned practice of a “mini-Q” assessment, it is sometimes preferable for the clinician to take signal measurements from one of the 10-20 locations on the adjacent sides of the midline axis (Fz-Cz-Pz-Oz). FIG. 3 shows the entire 19 sites for the International 10-20 system as well as the preauricular (M1/M2) areas for the attachment of a reference sensor. Measuring 19 sites at one time is a preferred embodiment of this system, but the present invention as designed should not be considered limited to this implementation, as other dry sensor systems may require or prefer alternate sensor locations or references setups (such as linked ears, common average, bipolar, and so on) for the type of signal analysis desired. FIG. 5 shows an example of another embodiment, a six sensor arrangement of the left frontal midline area using only the left earlobe/M1 location as a reference.

In the preferred system and method of the present invention, measurement validation is critical to insuring high signal quality and repeatable data. FIG. 4 shows an embodiment of a calibration setup which injects a series of sinusoid signals (401) into the input electrodes of the system and records the response curves for power, frequency response, phase error between channels. A calibration unit (402) evaluates the responses and determines if variables such as signal sampling rate, FFT algorithm, digital filtering or other selectable controls can be optimized. This type of functional test, routinely performed with conventional EEG devices, becomes essential to accepting the data it provides during human testing. A suitable example of a test signal generator is the TG315 Function Generator from Thurlby-Thandar. However, functionally it would need a modification to be compatible with some ultra-high impedance dry sensors. In addition to this type of testing, performed on a yearly basis to sanction the operation of the system, additional internal calibration testing shall be performed prior to subject testing. The selftest/calibration can be done before any client testing is done to insure response accuracy for the test system. If this built in test is successful than further client testing can proceed.

FIG. 6 shows one embodiment in which a client will be tested in a controlled setting, such as a school system screening program, professional sports team facility or a company sponsored test program. Upon entry into the test site (601) a number of screening procedures shall have been completed, which include but are not limited to: information provided to the client about the procedure and the informed consent from the client or their representative (parent, guardians). A determination that the client will receive some benefit from the procedure of gathering the EEG data, such as an evaluation for a condition, or a return to work indication. At this time it should also be predetermined what is the appropriate montage and number of sensors to be used for the testing process. When the client is seated for the beginning of testing, it would not be unusual to use a neck brace or other support to assist the client in remaining motionless during the testing procedure. (602) This method would be more appropriate for younger children who may need additional reassurance or instruction with the procedure at the start. It is also possible in one embodiment to use a pneumatic hand pump to fill the neck brace with air, and allow the child to control this pump to allow for a feeling of greater control and participation in the process. Following this initial explanation of the procedures, the client is asked to cooperate and generate a number of artifacts for the clinician performing the test, so that examples of the artifacts can be used to assist with a noise removal procedure. The client will be asked to generate eye blinks, up and down eye movements, jaw clenching, mouth twitching, swallowing, tongue motions and quiet lip movements (such as “YES”, “OK”).

At this point the client is comfortable and the dry sensors can be attached, normally through some sort of headset or helmet. The testing begins, (603) using trained operators or clinicians to make some initial pretests to establish a baseline electrical connection and measure the signal noise floor with at zero input. Then there is another collection of artifacts, to be stored in digital memory for later reference if necessary. In a preferred embodiment of the system, the typical acquisition shall consist of at least 60 to 120 intervals of from 2.0 to 4.0 seconds long of artifact free data. Acquisitions shall be done for both eyes closed and eyes opened conditions from the client. (604) In other embodiments, designed to focus on specific client needs or clinicians interests the intervals may be longer or shorter. At the conclusion of the test session a topographical summary of the test session will be provided on the computer used during the test to provide immediate visual test feedback as to the accuracy and validity of the data collection. The data can be reviewed and saved, and the session completed.

Finally the invention also encompasses program products comprising a computer readable file format, such as EDF or Lexicor, which is transportable to other mediums for translation. These embodiments, as listed above are not intended to be a limitation of the scope of the invention. The disclosed invention considers the embodiments presented as providing a variety of choices for algorithm selection, noise attenuation technique, signal processing method repertoire (window size, power spectrum measurement method, filtering method), as well as selection of the dry sensor implementation technology, in keeping with the spirit and form of the design.

### BRIEF DESCRIPTION OF THE DRAWINGS

FIG. 1 is a block diagram of a system of neurometric analysis using dry sensors on the client

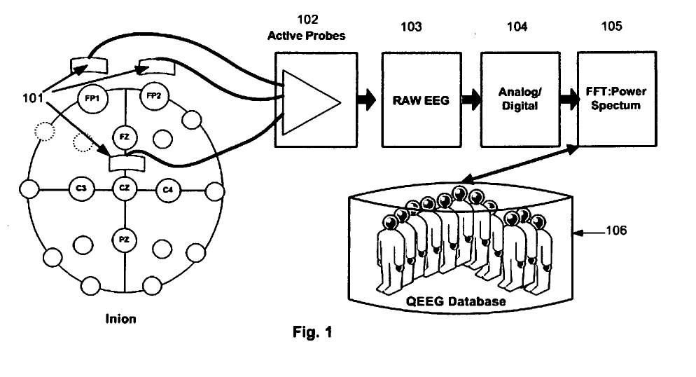

FIG. 2 is the primary (midline) and secondary locations targeted for acquisition by the dry sensors

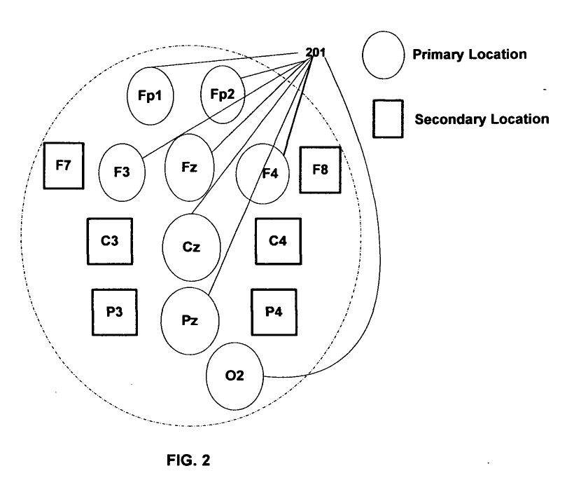

FIG. 3 shows a diagram of the International 10-20 system

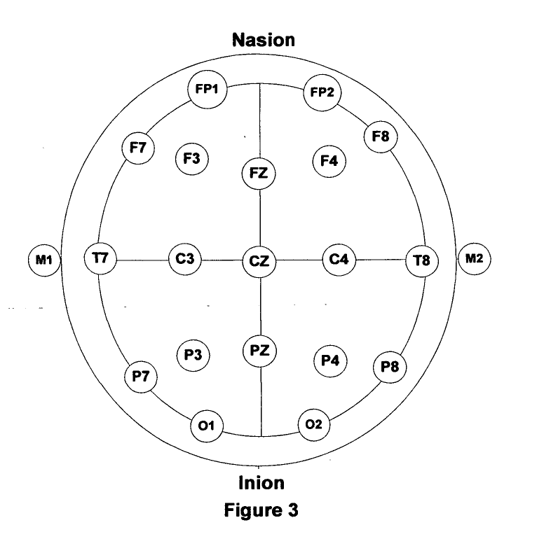

FIG. 4 is a diagram of an amplifier calibration system to measure system parameters and characterize the recording system

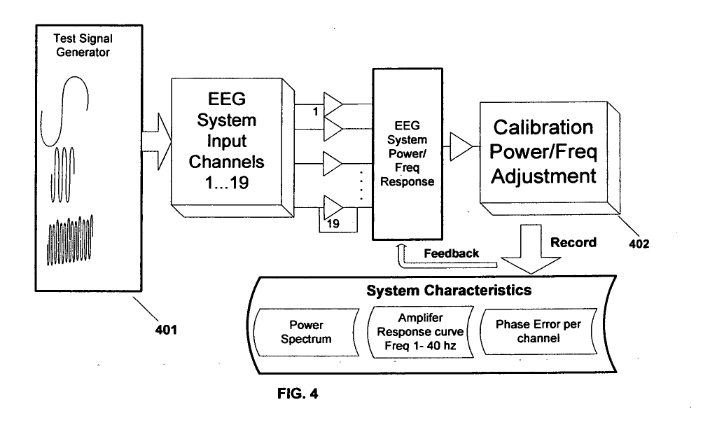

FIG. 5 is a montage suggesting one embodiment set up for a specific type of evaluation

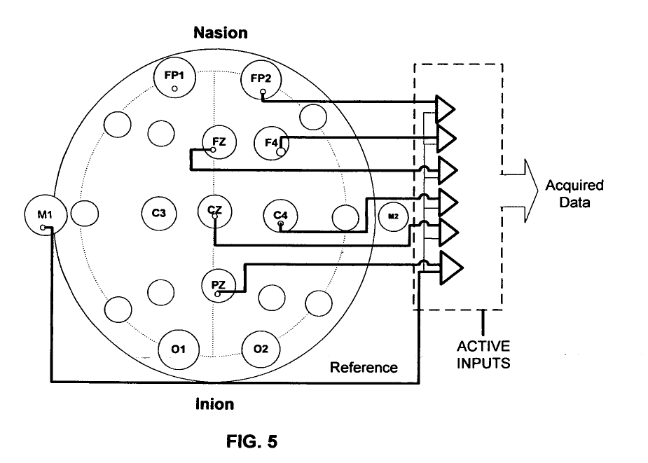

FIG. 6 is the activity flow of an acquisition session

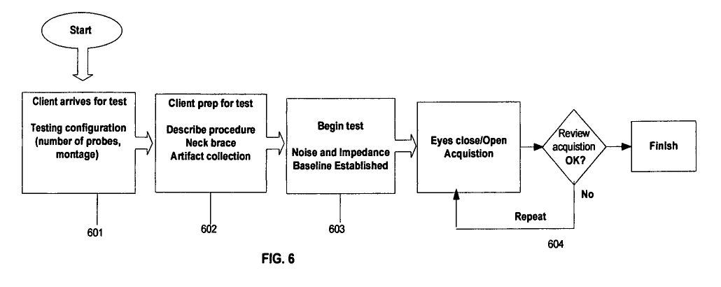

### OTHER PATENT REFERENCES

| | | |
|---|---|---|
| U.S. Pat. No. 3,744,482 | Jul. 10, 1973 | Dry Contact Electrode with Amplifier Physiological |
| | | Signals. William Kaufman, Donald P. Powell |
| U.S. Pat. No. 4,279,258 | Jul. 21, 1981 | Rapid Automatic EEG Evaluation |
| U.S. Pat. No. 4,411,273 | Oct. 25, 1983 | System and Method for Electrode Pair Derivation |
| U.S. Pat. No. 6,961,601 | Nov. 1, 2005 | Sensor System for Measuring Biopotentials |
| US2009/0312663 | Dec. 17, 2009 | System and Method for Neurometic Analysis |
| U.S. Pat. No. 7,754,190 B2 | Jul. 13, 2010 | Method for Determining Drug Effects Using |
| | | Quantitative EEG |
| US2011/0066065 | Mar. 17, 2011 | Systems and Methods to Identify a Subgroup of ADHD |
| | | at Higher Risk for Complicating Conditions. |
| US2011/0074396 | Mar. 31, 2011 | Biosensor and Electrode Structure Thereof |
| U.S. Pat. No. 8,454,505B2 | Feb. 7, 2012 | IMEC, Dry electrode technology |
| U.S. Pat. No. 8,112,139 | Feb. 7, 2012 | Skin Screw Electrode |
| US2012/0143020A1 | Jun. 7, 2012 | EEG Kit (EEG in a Bag) |
| U.S. Pat. No. 8,224,433 | Jul. 17, 2012 | Electroencephalography Based Systems and Methods |
| | | For Selecting Therapies and Predicting Outcomes |
| US20110310117 | Dec. 6, 2012 | Method for Diagnosing ADHD and Related Disorders |
| US2013/0023783A1 | Jan. 24, 2013 | NEBA EEG Assessment tool |

### OTHER REFERENCES

IFCN Standards IFCN standards for digital recording of Clinical EEG. Marc R. Nuwer, et al. Electroencephalography and clinical Neurophysiology 106 (1998) 259-261
Medical Bio Engineering Investigation into the origin of the noise of surface electrodes. E Huigen, A. Peper, C. A. Grimberger, Academic Medical Centre, University of Amsterdam, The Netherlands. 2002.
Child & Adolescent Psychiatric
Clinic of North America The role of quantitative electro encephalography in child and adolescent psychiatric disorders. Robert J. Chabot, Flavia di Michele, Leslie Prichep. 14(2005) 21-53
Internet Document Bristle-sensors-Dry Electrodes for EEG. Cristian Grozea, CatalinVonescuSiamacFazil, Fraunhofer Institute FIRST, Kekulestrasse 7 Berlin, German
Cigna Health Care Cigna Health Care Coverage Position, https://my.cigna.com/teamsite/health/provider/medical/procedural/coveraqe_positions/medical/mm_{—}0239_coveragepositioncriteria_quantitative_eeg_geeg.pdf, Nov. 15, 2006
Applied Neuroscience.com History of the Scientific Standards of QEEG Normative Databases Robert Thatcher and Joel Lubar. Published in: Introduction to QEEG and Neurofeedback: Advanced Theory and Applications, Thomas Budzinsky, H. Budzinsky, J. Evans, A. Abarbanel, Academic Press, San Diego, Calif. 2008
IEEE Trans Neural System
Rehabilitation Engineering Novel hydrogel-based preparation-free EEG electrode. Alba N A, Sclabassi R J, Sun M, Cu i XT, 2010 August; 18(4):415-23.
Army Research Laboratory Accounting for the Timing Drift of Variability in Contemporary EEG Systems. Dr. W. David Hairston, March 2012 ARL-TR-5945
Proceeding of the IEEE Biosensor Technologies for the Augmented Brain Computer Interfaces in the Next Decades, Lun-De Laio, Chin-Teng Lin, et all. May 13, 2012 Volume 100

We claim:

1. A method for assessing brain functioning, to promote and advance early screenings, said method comprising of 1) active electrode (also called dry sensor) technology for performing the signal acquisition, 2) at specific placement or predetermined placement locations on the scalp, and 3) incorporating Quantitative EEG statistical methods to derive prognostic data that will assist in planning treatments.

2. Using the method claimed in 1: Use of device for pre and post testing, and collection of EEG data for athletes in contact sports, including, but not limited to Football, hockey, wrestling, baseball, soccer at the high school, college and professional level.

3. Using the method claimed in 1: The use of this device to test young preschool children at risk for various types of behavioral, cognitive, learning and developmental delays: neurodevelopmental conditions including but not limited to Fetal alcohol spectrum disorder, Autism spectrum disorders, ADHD, Tourette's and other conditions.

4. Using the method claimed in 1: The use of this device to detect changes in the EEG related to performance enhancing training such as neurofeedback training for executive functioning, sensory motor conditions, OT (Occupational therapy treatment), as well as personal professional counseling, or other therapeutic treatments such as relaxation training and physiological biofeedback. And to evaluate the treatments as to their efficacy for the user.

5. Using the method claimed in 1: The use of this device to detect changes in the EEG related to chronic pain conditions including the following but not limited to such as migraine headaches, tension headaches, chronic fatigue syndrome, anxiety disorders and fibromyalgia. Additionally, using the findings for providing interventions/neurofeedback trainings. And to evaluate the treatments as to their pre and post efficacy of treatment for the user.

6. Using the method claimed in 1: The use of this device to detect changes in the EEG related to the following conditions: post traumatic stress disorders, anxiety & sleep disorders. Additionally, using the findings for providing interventions/neurofeedback trainings. And to evaluate the treatments as to their pre and post treatment efficacy for the user.

7. Using the method claimed in 1: The use of this device to detect changes in the EEG related to the neurological conditions including the following but not limited to: dementia, stroke, multiple sclerosis, epilepsy etc. Additionally, using the findings for providing interventions/neurofeedback trainings. And to evaluate the treatments as to their pre and post treatment efficacy for the user.

8. Using the method claimed in 1: The use of this device to detect changes in the EEG related to various mental health, medical and academic conditions in special education, juvenile justice, corrections, foster care, adoption and employment screening. Additionally, using the findings for providing interventions/neurofeedback trainings. And to evaluate the treatments as to their pre and post treatment efficacy for the user.

9. Using the method claimed in 1: The use of this device to detect changes in the EEG related to regiments of prescription drugs that are used to improve mental health, psychosocial, medical, neurological, cognitive, behavioral, developmental and emotional conditions. And to evaluate the prescription as to their pre and post treatment efficacy for the user.

10. Using the method claimed in 1: The use of this device to detect preexisting conditions for the procurement of insurance policies, or as an adjunct to evaluate as a baseline basic indices of mental, medical & cognitive health.

11. Using the method claimed in 1: The use of this device to collect data of pre and post QEEG guided neurofeedback treatments and interventions for the sole purpose of getting insurance companies to approve this (QEEG guided neurofeedback treatments) procedure and determine reimbursement rates.

12. Using the method claimed in 1: The use of this device to detect preexisting conditions for the admission of individuals into the United State Military Armed Forces, or as an adjunct to evaluate as a baseline basic indices of cognitive health.

13. Using the method claimed in 1: Use of this device as a office based and cost effective way to augment the analysis of optimal brain functioning of such additional diagnostic tools such as functional Magnetic Resonance Imaging, and other imaging technologies such as Diffusion tensor imaging, Computer Tomography, Single Photon emission computer tomography (SPECT)

14. Using the method claimed in 1: Use of this device to assist in the development of specific types of population and symptom based QEEG databases for the purpose of improved symptom identification and the development of integrated treatments.

15. Using the method claimed in 1: The use of this device to detect changes in the EEG related to the substance abuse disorders and addiction issues. Additionally, using the findings for providing interventions/Neurofeedback trainings. And to evaluate the treatments as to their pre and post treatment efficacy for the user.

16. What is further claimed: A method for assessing brain functioning, to promote and advance early screenings, said method comprising of 1) active electrode (also called dry sensor) technology for performing the signal acquisition, 2) at specific and predetermined placement locations on the scalp, and 3) incorporating Quantitative EEG statistical methods with eLORETA source location techniques to derive diagnostic data.
